(19) United States
(12) Patent Application Publication
(10) Pub. No.: US 2013/0338415 A1
(43) Pub. Date: Dec. 19, 2013

(54) PROCESS FOR THE CONVERSION OF MIXED LOWER ALKANES TO AROMATIC HYDROCARBONS

(71) Applicants: Mahesh Venkataraman Iyer, Cypress, TX (US); Ann Marie Lauritzen, Houston, TX (US); Ajay Madhav Madgavkar, KAty, TX (US); NIck Joseph Vecchio, Friendswood, TX (US)

(72) Inventors: Mahesh Venkataraman Iyer, Cypress, TX (US); Ann Marie Lauritzen, Houston, TX (US); Ajay Madhav Madgavkar, KAty, TX (US); NIck Joseph Vecchio, Friendswood, TX (US)

(21) Appl. No.: 13/991,458

(22) PCT Filed: Dec. 5, 2011
(86) PCT No.: PCT/US11/63286

§ 371 Date: Sep. 4, 2013

Related U.S. Application Data
(60) Provisional application No. 61/420,044, filed on Dec. 6, 2010.

(51) Int. Cl. C07C 2/76

(52) U.S. Cl. CPC C07C 2/76
USPC 585/322

(57) ABSTRACT

A process comprising: contacting a lower alkane feed comprising propane and ethane with an aromatization catalyst in a first stage under first stage reaction conditions to produce a first stage product stream comprising ethane and aromatics; separating the aromatics from the first stage product stream to form an aromatics product stream and a second stage feed; and contacting the second stage feed with an aromatization catalyst in a second stage under second stage reaction conditions to produce a second stage product stream comprising ethane and aromatics wherein the amount ethane in the first stage product stream is equal to from 80 to 300% of the amount of ethane in the lower alkane feed and the amount of ethane in the second stage product stream is 500 equal to at most 80% of the amount of ethane in the second stage feed is described.

### FIELD OF THE INVENTION

The present invention relates to a process for producing aromatic hydrocarbons from mixed lower alkanes. More specifically, the invention relates to a two stage process for increasing the production of benzene from a mixture of ethane and propane or ethane, propane and butane in a dehydroaromatization process.

### BACKGROUND OF THE INVENTION

There is a projected global shortage for benzene which is needed in the manufacture of key petrochemicals such as styrene, phenol, nylon and polyurethanes, among others. Generally, benzene and other aromatic hydrocarbons are obtained by separating a feedstock fraction which is rich in aromatic compounds, such as reformate produced through a catalytic reforming process and pyrolysis gasolines produced through a naphtha cracking process, from non-aromatic hydrocarbons using a solvent extraction process.

To meet this projected supply shortage, numerous catalysts and processes for on-purpose production of aromatics (including benzene) from alkanes containing six or less carbon atoms per molecule have been investigated. These catalysts are usually bifunctional, containing a zeolite or molecular sieve material to provide acidity and one or more metals such as Pt, Ga, Zn, Mo, etc. to provide dehydrogenation activity. For example, U.S. Pat. No. 4,350,835 describes a process for converting ethane-containing gaseous feeds to aromatics using a crystalline zeolite catalyst of the ZSM-5-type family containing a minor amount of Ga. As another example, U.S. Pat. No. 7,186,871 describes aromatization of C₁-C₄ alkanes using a catalyst containing Pt and ZSM-5.

Most lower alkane dehydroaromatization processes carry out the reaction in one step. For example, EP0147111 describes an aromatization process wherein a C₃-C₄ feed is mixed with ethane and all are reacted together in a single reactor. A minority of these processes involves two separate steps or stages. For example, U.S. Pat. No. 3,827,968 describes a process which involves oligomerization followed by aromatization. U.S. Pat. No. 4,554,393 and U.S. Pat. No. 4,861,932 describe two-step processes for propane involving dehydrogenation followed by aromatization. None of these examples mention a two-stage process in which lower alkane aromatization takes place in both stages.

The ease of conversion of individual alkanes to aromatics increases with increasing carbon number. When a mixed feed consisting of ethane and higher hydrocarbons is converted into benzene plus higher aromatics in a single stage, the selection of reaction severity is dictated by the desired overall hydrocarbon conversion target. If a significant level of ethane conversion is desired or needed, this may lead to operating such a one-stage process at higher temperature severity. The negative consequence of this higher severity is such that higher hydrocarbons such as propane can undergo non-selective side reactions which result in excessive hydrogenolysis into lower-valued methane. The net result is that the overall yield to benzene and other aromatics is reduced significantly.

It would be advantageous to provide a light alkane dehydroaromatization process wherein (a) the conversion of each component of a mixed alkane feed can be optimized, (b) the ultimate yield of benzene is greater than that of any other single aromatic product, and (c) the generation of undesired methane by-product is minimized.

### SUMMARY OF THE INVENTION

The above problem is resolved according to the present invention by using the ethane to improve the selectivity to aromatics achieved during a first stage and then converting the ethane to aromatics in a second stage. This is accomplished by designing a two-stage process as described below.

The present invention provides a process comprising: a.) contacting a lower alkane feed comprising propane and ethane with an aromatization catalyst in a first stage under first stage reaction conditions to produce a first stage product stream comprising ethane and aromatics; b.) separating the aromatics from the first stage product stream to form an aromatics product stream and a second stage feed; and c.) contacting the second stage feed with an aromatization catalyst in a second stage under second stage reaction conditions to produce a second stage product stream comprising ethane and aromatics wherein the amount of ethane in the first stage product stream is equal to from 80 to 300% of the amount of ethane in the lower alkane feed and the amount of ethane in the second stage product stream is equal to at most 80% of the amount of ethane in the second stage feed.

Fuel gas, which includes primarily methane and hydrogen, may also be produced in either or both of the first and second stages. The fuel gas may be separated from the aromatic reaction products in either or both of the stages. Thus, fuel gas may be an additional product of the process of this invention.

### BRIEF DESCRIPTION OF THE DRAWINGS

FIG. 1 is a schematic flow diagram which illustrates the process scheme for producing aromatics (benzene and higher aromatics) from a mixed lower alkane feed containing at least ethane and propane using a one reactor-regenerator stage process. FIG. 2 is a schematic flow diagram for producing aromatics (benzene and higher aromatics) from a mixed lower alkane feed containing at least ethane and propane using a two stage reactor-regenerator system.

### DETAILED DESCRIPTION OF THE INVENTION

The present invention is a process for producing aromatic hydrocarbons which comprises bringing into contact a hydrocarbon feedstock containing at least propane and ethane, preferably at least 20 wt % ethane and at least 20 wt % propane, and possibly other hydrocarbons such as butane, and a catalyst composition suitable for promoting the reaction of such hydrocarbons to aromatic hydrocarbons, such as benzene, at a temperature of from about 400 to about 700 C and a pressure of from about 0.01 to about 1.0 Mpa absolute. The gas hourly space velocity (GHSV) per hour may range from about 300 to about 6000. These conditions are used in each of the stages but the conditions in the stages may be the same or different. The conditions may be optimized for the conversion of propane, and possibly other higher alkanes such as butane, in the first stage and ethane in the second stage. In the first stage, the reaction temperature preferably ranges from about 400 to about 650 C, more preferably from about 420 to about 650 C and most preferably from about 480 to 600° C. In the second stage, the reaction temperature preferably ranges from about 450 to about 680 C, more preferably from about 450 to about 675 C and most preferably from 575 to 675° C. The primary desired products of the process of this invention are benzene, toluene and xylene (BTX). In an embodiment, the first stage reaction conditions may be optimized for the conversion of propane to BTX. Optionally, the first stage reaction conditions may also be optimized for the conversion of any higher hydrocarbons which may be present in the feedstock to BTX. In another embodiment, the second stage reaction conditions may be optimized for the conversion of ethane to BTX. Optionally, the second stage reaction conditions may also be optimized for the conversion of any other non-aromatic hydrocarbons which may be produced in the first stage to BTX.

The first stage and second stage reactors may be operated under similar conditions. When either reactor is run at higher temperatures, i.e., above about 630-650 C, more fuel gas and less aromatics are produced even though the net feed conversion per pass for that stage may be higher. Therefore it is better to run at lower temperature and convert less feed in each pass of each stage in order to produce more aromatics in total even though more ethane will have to be recycled. Operating in the preferred range helps to maximize aromatics production by minimizing fuel gas production. The use of higher temperatures may maximize the production of fuel gas.

The first stage may be operated such that the amount of ethane in the outlet from the first stage comprises 80 to 300% of the amount of ethane in the inlet to the first stage. The ethane in the outlet may be from 150 to 300% of the ethane in the inlet. In another embodiment, the ethane in the outlet may be from 200 to 300% of the ethane in the inlet.

The second stage may be operated such that the amount of ethane in the outlet from the second stage comprises at most 80% of the amount of ethane in the inlet to the second stage. The ethane in the outlet may comprise at most 70% of the ethane in the inlet. In another embodiment, the ethane in the outlet may comprise at most 60% of the ethane in the inlet. The ethane in the outlet may comprise from 5 to 80% of the ethane in the inlet, from 10 to 70% or from 20 to 60% of the ethane in the inlet.

In another embodiment, second stage may be operated such that the amount of ethane in the outlet from the second stage comprises at most 50% of the amount of ethane in the inlet to the second stage. The ethane in the outlet may comprise at most 40% of the ethane in the inlet. In another embodiment, the ethane in the outlet may comprise at most 30% of the ethane in the inlet.

Fuel gas may be an additional product of the process of the present invention. Fuel gas includes primarily methane and hydrogen which are produced along with the aromatics. Fuel gas may be used for power and/or steam generation. The hydrogen in the fuel gas may be separated and used for refinery or chemical reactions that require hydrogen, including the hydrodealkylation of toluene and/or xylene as discussed below.

It is possible to carry out this process in batch mode using separate reactors for each stage or using the same reactor for each stage but it is highly preferred that it be carried out in continuous mode in separate reactors. Each stage may be carried out in a single reactor or in two or more reactors aligned in parallel. Preferably, at least two reactors are used in each stage so that one reactor may be in use for aromatization while the other reactor is offline so the catalyst may be regenerated. The aromatization reactor system may be a fluidized bed, moving bed or a cyclic fixed bed design. The cyclic fixed bed design is preferred for use in this invention.

The hydrocarbons in the feedstock may be comprised of at least about 20 wt % of propane, at least about 20 wt % of ethane, and, optionally, at least about 10 to 20 wt % of butane, pentane, etc. In one embodiment, the feedstock is from about 30 to about 50 wt % propane and from about 30 to about 50 wt % ethane. The feed may contain small amounts of C₂-C₄ olefins, preferably no more than 5 to 10 weight percent. Too much olefin may cause an unacceptable amount of coking and deactivation of the catalyst.

A mixed propane/ethane or mixed C₂-C₄ lower alkane feed stream may be derived from, for example, an ethane/propane-rich stream derived from natural gas, refinery or petrochemical streams including waste streams. Examples of potentially suitable feed streams include (but are not limited to) residual ethane and propane and butane from natural gas (methane) purification, pure ethane and propane and butane streams (also known as Natural Gas Liquids) co-produced at a liquefied natural gas (LNG) site, C₂-C₄ streams from associated gases co-produced with crude oil production (which are usually too small to justify building a LNG plant but may be sufficient for a chemical plant), unreacted “waste” streams from steam crackers, and the C₁-C₄ byproduct stream from naphtha reformers (the latter two are of low value in some markets such as the Middle East).

Usually natural gas, comprising predominantly methane, enters an LNG plant at elevated pressures and is pre-treated to produce a purified feed stock suitable for liquefaction at cryogenic temperatures. Ethane, propane, butane and other gases are separated from the methane. The purified gas (methane) is processed through a plurality of cooling stages using heat exchangers to progressively reduce its temperature until liquefaction is achieved. The separated gases may be used as the feed stream of the present invention. The byproduct streams produced by the process of the present invention may have to be cooled for storage or recycle and the cooling may be carried out using the heat exchangers used for the cooling of the purified methane gas.

Any one of a variety of catalysts may be used to promote the reaction of propane and ethane and possibly other alkanes to aromatic hydrocarbons. One such catalyst is described in U.S. Pat. No. 4,899,006 which is herein incorporated by reference in its entirety. The catalyst composition described therein comprises an aluminosilicate having gallium deposited thereon and/or an aluminosilicate in which cations have been exchanged with gallium ions. The molar ratio of silica to alumina is at least 5:1.

Another catalyst which may be used in the process of the present invention is described in EP 0 244 162. This catalyst comprises the catalyst described in the preceding paragraph and a Group VIII metal selected from rhodium and platinum. The aluminosilicates are said to preferably be MFI or MEL type structures and may be ZSM-5, ZSM-8, ZSM-11, ZSM-12 or ZSM-35.

Other catalysts which may be used in the process of the present invention are described in U.S. Pat. No. 7,186,871 and U.S. Pat. No. 7,186,872, both of which are herein incorporated by reference in their entirety. The first of these patents describes a platinum containing ZSM-5 crystalline zeolite synthesized by preparing the zeolite containing the aluminum and silicon in the framework, depositing platinum on the zeolite and calcining the zeolite. The second patent describes such a catalyst which contains gallium in the framework and is essentially aluminum-free.

It is preferred that the catalyst be comprised of a zeolite, a noble metal of the platinum family to promote the dehydrogenation reaction, and a second inert or less active metal which will attenuate the tendency of the noble metal to catalyze hydrogenolysis of the C₂ and higher hydrocarbons in the feed to methane and/or ethane. Attenuating metals which can be used include those described below.

Additional catalysts which may be used in the process of the present invention include those described in U.S. Pat. No. 5,227,557, hereby incorporated by reference in its entirety. These catalysts contain an MFI zeolite plus at least one noble metal from the platinum family and at least one additional metal chosen from the group consisting of tin, germanium, lead, and indium.

One preferred catalyst for use in this invention is described in U.S. Patent Application Publication No. 2009/0209795. This publication is hereby incorporated by reference in its entirety. The publication describes a catalyst comprising: (1) 0.005 to 0.1 wt % (% by weight) platinum, based on the metal, preferably 0.01 to 0.05 wt %, (2) an amount of an attenuating metal selected from the group consisting of tin, lead, and germanium which is preferably not more than 0.2 wt % of the catalyst, based on the metal and wherein the amount of platinum may be no more than 0.02 wt % more than the amount of the attenuating metal; (3) 10 to 99.9 wt % of an aluminosilicate, preferably a zeolite, based on the aluminosilicate, preferably 30 to 99.9 wt %, preferably selected from the group consisting of ZSM-5, ZSM-11, ZSM-12, ZSM-23, or ZSM-35, preferably converted to the H+ form, preferably having a SiO₂/Al₂O₃ molar ratio of from 20:1 to 80:1, and (4) a binder, preferably selected from silica, alumina and mixtures thereof.

Another preferred catalyst for use in this invention is described in U.S. patent application Ser. No. 12/867973, filed Aug. 17, 2010. This application is hereby incorporated by reference in its entirety. The application describes a catalyst comprising: (1) 0.005 to 0.1 wt % (% by weight) platinum, based on the metal, preferably 0.01 to 0.06 wt %, most preferably 0.01 to 0.05 wt %, (2) an amount of iron which is equal to or greater than the amount of the platinum but not more than 0.50 wt % of the catalyst, preferably not more than 0.20 wt % of the catalyst, most preferably not more than 0.10 wt % of the catalyst, based on the metal; (3) 10 to 99.9 wt % of an aluminosilicate, preferably a zeolite, based on the aluminosilicate, preferably 30 to 99.9 wt %, preferably selected from the group consisting of ZSM-5, ZSM-11, ZSM-12, ZSM-23, or ZSM-35, preferably converted to the H+form, preferably having a SiO₂/Al₂O₃ molar ratio of from 20:1 to 80:1, and (4) a binder, preferably selected from silica, alumina and mixtures thereof.

Another preferred catalyst for use in this invention is described in U.S. Patent Application Publication No. 2009/0209794. This publication is hereby incorporated by reference in its entirety. The publication describes a catalyst comprising: (1) 0.005 to 0.1 wt % (% by weight) platinum, based on the metal, preferably 0.01 to 0.05% wt, most preferably 0.02 to 0.05% wt, (2) an amount of gallium which is equal to or greater than the amount of the platinum, preferably no more than 1 wt %, most preferably no more than 0.5 wt %, based on the metal; (3) 10 to 99.9 wt % of an aluminosilicate, preferably a zeolite, based on the aluminosilicate, preferably 30 to 99.9 wt %, preferably selected from the group consisting of ZSM-5, ZSM-11, ZSM-12, ZSM-23, or ZSM-35, preferably converted to the H+ form, preferably having a SiO₂/Al₂O₃ molar ratio of from 20:1 to 80:1, and (4) a binder, preferably selected from silica, alumina and mixtures thereof.

One of the undesirable products of the aromatization reaction is coke which may deactivate the catalyst. While catalysts and operating conditions and reactors are chosen to minimize the production of coke, it is usually necessary to regenerate the catalyst at some time during its useful life. Regeneration may increase the useful life of the catalyst. Regeneration of coked catalysts has been practiced commercially for decades and various regeneration methods are known to those skilled in the art.

The regeneration of the catalyst may be carried out in the aromatization reactor or in a separate regeneration vessel or reactor. For example, the catalyst may be regenerated by burning the coke at high temperature in the presence of an oxygen-containing gas as described in U.S. Pat. No. 4,795,845 which is herein incorporated by reference in its entirety. Regeneration with air and nitrogen is shown in the examples of U.S. Pat. No. 4,613,716 which is herein incorporated by reference in its entirety. Another possible method involves air calcination, hydrogen reduction, and treatment with sulfur or a sulfurization material. Platinum catalysts have been used to assist the combustion of coke deposited on such catalysts.

The preferred regeneration temperature range for use herein is from about 450 to about 788 C. The preferred temperature range for regeneration in the first stage is from about 470 to about 788 C. The preferred temperature range for regeneration in the second stage is from about 500 to about 788 C.

The unreacted methane and byproduct may be used in other steps, stored and/or recycled. It may be necessary to cool these byproducts to liquefy them. When the ethane or mixed lower alkanes originate from an LNG plant as a result of the purification of the natural gas, at least some of these byproducts may be cooled and liquefied using the heat exchangers used to liquefy the purified natural gas (methane).

The toluene and xylene may be converted into benzene by hydrodealkylation. The hydrodealkylation reaction involves the reaction of toluene, xylenes, ethylbenzene, and higher aromatics with hydrogen to strip alkyl groups from the aromatic ring to produce additional benzene and light ends including methane and ethane which are separated from the benzene. This step substantially increases the overall yield of benzene and thus is highly advantageous.

Both thermal and catalytic hydrodealkylation processes are known in the art. Methods for hydrodealkylation are described in U.S. Patent Application Publication No. 2009/0156870 which is herein incorporated by reference in its entirety.

The integrated process of this invention may also include the reaction of benzene with propylene to produce cumene which may in turn be converted into phenol and/or acetone. The propylene may be produced separately in a propane dehydrogenation unit or may come from olefin cracker process vent streams or other sources. Methods for the reaction of benzene with propylene to produce cumene are described in U.S. Patent Application Publication No. 2009/0156870 which is herein incorporated by reference in its entirety.

The integrated process of this invention may also include the reaction of benzene with olefins such as ethylene. The ethylene may be produced separately in an ethane dehydrogenation unit or may come from olefin cracker process vent streams or other sources. Ethylbenzene is an organic chemical compound which is an aromatic hydrocarbon. Its major use is in the petrochemical industry as an intermediate compound for the production of styrene, which in turn is used for making polystyrene, a commonly used plastic material. Methods for the reaction of benzene with ethylene to produce ethylbenzene are described in U.S. Patent Application Publication No. 2009/0156870 which is herein incorporated by reference in its entirety.

Styrene may then be produced by dehydrogenating the ethylbenzene. One process for producing styrene is described in U.S. Pat. No. 4,857,498, which is herein incorporated by reference in its entirety. Another process for producing styrene is described in U.S. Pat. No. 7,276,636, which is herein incorporated by reference in its entirety.

### EXAMPLES

The following examples are provided for illustrative purposes only and are not intended to limit the scope of the invention.

### Example 1

In this example the results of laboratory tests are used to represent the improvements in total aromatics yield that may be obtained by using ethane in both stages of the two-stage lower alkane aromatization process of the present invention. The laboratory tests of this example illustrate the aromatics yields that can be obtained with a single-stage aromatization process using 100% wt propane feed, a single-stage aromatization process using a feed consisting of 19.9% wt ethane and 80.1% wt propane, a two-stage aromatization process in which the first-stage feed consists of 100% wt propane and the second-stage feed consists of the ethane byproduct from the first stage, and the two-stage aromatization process of the present invention in which the first-stage feed consists of 19.9% wt ethane and 80.1% wt propane and the second-stage feed consists of the net ethane produced in the first stage.

Catalyst A was made on 1.6 mm diameter cylindrical extrudate particles containing 80% wt of zeolite ZSM-5 CBV 2314 powder (23:1 molar SiO₂/Al₂O₃ ratio, available from Zeolyst International) and 20% wt alumina binder. The extrudate samples were calcined in air up to 650° C. to remove residual moisture prior to use in catalyst preparation. The target metal loadings for Catalyst A were 0.025% w Pt and 0.09% wt Ga.

Metals were deposited on 25-100 gram samples of the above ZSM-5/alumina extrudate by first combining appropriate amounts of stock aqueous solutions of tetraammine platinum nitrate and gallium(III) nitrate, diluting this mixture with deionized water to a volume just sufficient to fill the pores of the extrudate, and impregnating the extrudate with this solution at room temperature and atmospheric pressure. Impregnated samples were aged at room temperature for 2-3 hours and then dried overnight at 100° C. Fresh 15-cc charges of Catalyst A were subjected to performance tests as described below. For each performance test, a 15-cc charge of fresh (not previously tested) catalyst was loaded “as is,” without crushing, into a Type 316H stainless steel tube (1.40 cm i.d.) and positioned in a four-zone furnace connected to a gas flow system. Prior to each performance test, the fresh charge of Catalyst A was pretreated in situ at atmospheric pressure (about 0.1 MPa absolute) as follows:
(a) calcination with air at approximately 60 liters per hour (L/hr), during which the reactor wall temperature was raised from 25 to 510° C. in 12 hrs, held at 510° C. for 4 hrs, then further increased from 510° C. to the target reactor wall temperature for the run in 1 hr, then held at the target run temperature with continued air flow for 30 min;
(b) nitrogen purge at approximately 60 L/hr, at the target run temperature, for 20 min;
(c) reduction with hydrogen at 60 L/hr, at the target run temperature, for 30 min.

At the end of the above reduction step, the hydrogen flow was terminated, and the catalyst charge was exposed to the indicated feed at the target run temperature, atmospheric pressure (about 0.1 MPa absolute), and a total feed rate of 1000 GHSV (1000 cc feed per cc of catalyst per hr). Three minutes after introduction of the feed, the total reactor outlet stream was sampled by an online gas chromatograph for analysis.

Table 1 lists the feed compositions, reactor wall temperatures, and results of online gas chromatographic analyses of the total product streams from Performance Tests 1 through 3 that were conducted with fresh charges of Catalyst A as described above. Based on composition data obtained from the gas chromatographic analyses, initial ethane and propane conversions were computed according to the formulas given below:

Ethane conversion, %=100×(% wt ethane in feed−% wt ethane in outlet stream)/(% wt ethane in feed)

Propane conversion, %=100×(% wt propane in feed−% wt propane in outlet stream)/(% wt propane in feed)

For Performance Tests 1 and 2, normalized % wt yields, based on propane feed converted, for each component, except ethane, in the reactor outlet stream were computed according to the following formula:

Normalized % wt yield of component C=10,000×(% wt component C in reactor outlet stream)/(% wt propane in feed×% propane conversion)

For Performance Tests 1 and 2, the normalized % wt net ethane yield, based on propane feed converted, was computed according to the following formula:

Normalized % wt net ethane yield=10,000×(% wt ethane in reactor outlet stream−% wt ethane in feed)/(% wt propane in feed×% propane conversion)

For Performance Test 3, normalized % wt yields, based on ethane feed converted, for each component, except ethane, in the reactor outlet stream were computed according to the following formula:

Normalized % wt yield of component C=100×(% wt component C in reactor outlet stream)/(% wt ethane conversion)

TABLE 1

| | Performance Test No. | | |
|---|---|---|---|
| | 1 | 2 | 3 |
| Reactor Wall Temperature, C. | 600 | 600 | 621 |
| Feed Ethane, % wt | 0 | 19.9 | 100 |
| Feed Propane, % wt | 100 | 80.1 | 0 |
| Ethane Conversion, % wt | NA | −99.81 | 48.34 |
| Propane Conversion, % wt | 99 | 99.25 | NA |
| Reactor Outlet Composition, % wt | | | |
| Hydrogen | 3.03 | 3.79 | 4.56 |
| Methane | 22.18 | 12.64 | 7.94 |
| Ethylene | 1.8 | 2.27 | 4.37 |
| Ethane | 28.84 | 39.71 | 51.66 |
| Propylene | 0.53 | 0.37 | 0.57 |
| Propane | 1 | 0.6 | 0.63 |
| C4 | 0.07 | 0.06 | 0.11 |
| C5 | 0 | 0 | 0 |
| Benzene | 19.56 | 18.68 | 14.61 |
| Toluene | 13.26 | 12.47 | 6.94 |
| C8 Aromatics | 2.73 | 2.55 | 1.19 |
| C9+ Aromatics | 6.99 | 6.85 | 7.41 |
| Total Aromatics | 42.54 | 40.55 | 30.15 |
| Normalized Total Aromatics Yield | 42.97 | 51.01 | |
| Based on Propane Converted | | | |
| Normalized Ethane Yield | 29.13 | 24.92 | |
| Based on Propane Converted | | | |
| Normalized Total Aromatics Yield Based on | | | 62.37 |
| Ethane Converted in Stage 2 (Test 3) | | | |
| Added Total Aromatics from Stage 2 | 18.16 | 15.54 | |
| Normalized Total Aromatics Yield, | 61.13 | 66.55 | |
| Stages 1 and 2 | | | |

From the results for Performance Tests 1 and 2 in Table 1, it can be seen that the normalized total aromatics yields obtained, based on propane converted, were 42.97% wt for the feed consisting of 100% wt propane and 51.01% wt for the feed consisting of 19.9% wt ethane plus 80.1% wt propane. These results simulate the benefit of including ethane in the feed for a one-stage propane aromatization process.

From the results for Performance Test 1 in Table 1, it can also be seen that the normalized ethane yield, based on propane converted, obtained from the feed consisting of 100% wt propane was 29.13% wt. If all of the ethane byproduct from Performance Test 1 was taken through a second conversion stage, simulated by Performance Test 3 which was conducted with 100% wt ethane feed, then the normalized total aromatics yield obtained from this second stage, based on the original amount of propane fed to the first stage, would be 18.16% wt. Thus, the total combined normalized aromatics yield, based on original propane feed, from the first stage fed with 100% propane and the second stage fed with byproduct ethane from the first stage would be the sum of 42.97% wt from the first stage plus 18.16% wt from the second stage, for a total normalized aromatics yield of 61.13% wt. These results illustrate the benefit of converting ethane byproduct from the first stage of a propane aromatization process in a second stage, with the second stage providing a substantial increase in the total aromatics yield derived from the original propane feed.

From the results for Performance Test 2 in Table 1, it can be seen that the normalized ethane, based on propane converted, obtained from the feed consisting of 19.9% wt ethane plus 80.1% wt propane was 24.92% wt. If all of the net ethane byproduct from Performance Test 2 was taken through a second conversion stage, simulated by Performance Test 3 which was conducted with 100% wt ethane feed, then the normalized total aromatics yield obtained from this second stage, based on the original amount of propane fed to the first stage, would be 15.54% wt. Thus, the total combined normalized aromatics yield, based on original propane feed, from the first stage fed with 19.9% wt ethane plus 80.1% wt propane, and the second stage fed with byproduct ethane from the first stage, would be the sum of 51.01% wt from the first stage plus 15.54% wt from the second stage, for a total normalized aromatics yield of 66.55% wt. This 66.55% wt total normalized aromatics yield clearly exceeds the 42.97% wt value obtained from one-stage processing of 100% wt propane feed (simulated by Performance Test 1), the 51.01% wt value obtained from one-stage processing of the feed consisting of 19.9% wt ethane plus 80.1% wt propane (simulated by Performance Test 2), and the 61.13% wt value obtained from two-stage processing in which the first-stage feed consists of 100% wt propane and the second-stage feed consists of the net ethane byproduct from the first stage (simulated by Performance Tests 1 and 3, respectively). Thus, these results illustrate the benefit of the two-stage lower alkane aromatization process comprised by the present invention, in which ethane is present as a feed component but not converted in the first aromatization stage, and in which ethane byproduct from the first stage is converted in the second aromatization stage.

### Example 2

2.1 Two-Stage Process with no ethane in first stage feed

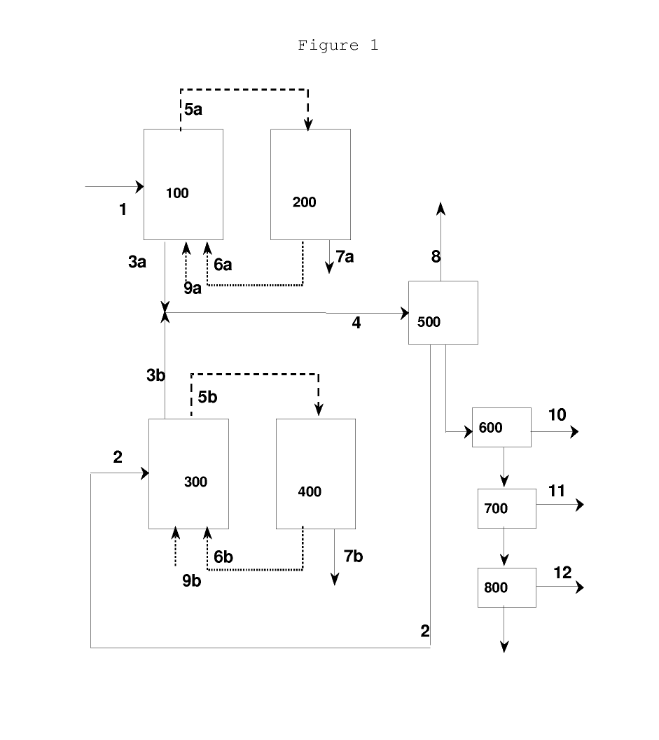

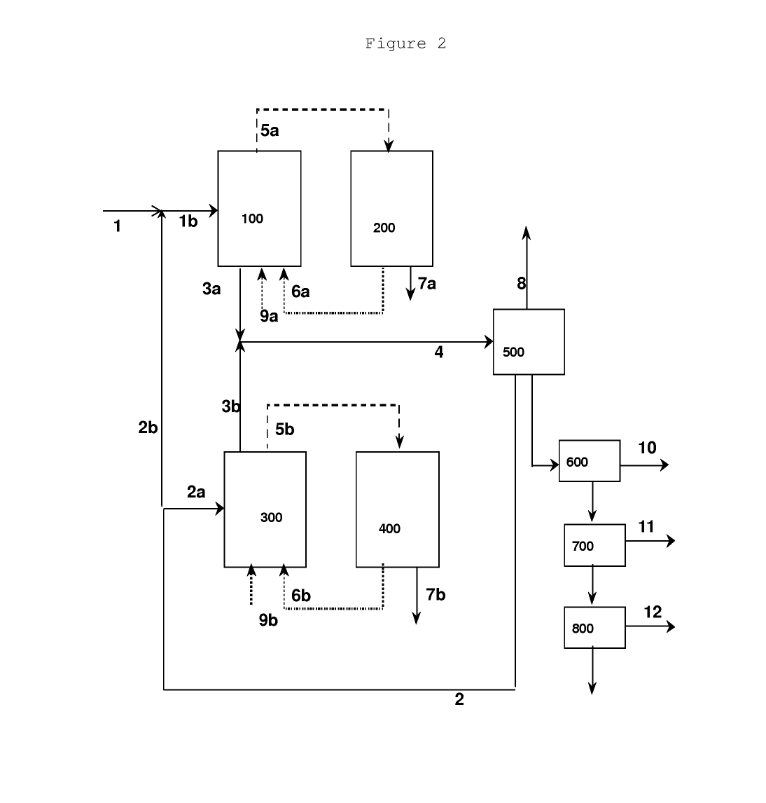

FIG. 1 is a schematic flow diagram for producing aromatics (benzene and higher aromatics) from a feed. This example demonstrates the results of feeding a feedstream containing 100 wt % propane using a two stage reactor-regenerator system.

25 tonnes/hr (tph) of feed (stream 1), which constitutes primarily 100 wt % propane including minor amounts of methane, ethane, butane, etc. (stream 1) is fed to the stage 1 aromatization reactor 100 that uses “Catalyst A” described in example 1. The first stage reactor 100 operates at about 1 atmosphere pressure and at a temperature of about 600° C. while the stage 1 regenerator 200, which removes the coke formed in the reactor 100, operates at around 730° C. The heat required for the reaction step is provided by the hot catalyst solid mixture which is preheated during the regeneration step. The reactor 100 achieves almost complete conversion of propane with a negative value recorded for ethane conversion as shown in Table 1 for Performance Test 1 since ethane is made as a byproduct of propane conversion converted in this test. The reactor effluent stream 3a is then mixed with the reactor effluent from the second stage reactor 300 (stream 3b), described below. The combined effluent from both the reactor stages (stream 4) is then fed to a separation system where unconverted reactants and light hydrocarbons that consist primarily of ethane and some other hydrocarbons, which may include ethylene, propane, propylene, methane, butane and some hydrogen, are used as the feed (stream 2) for the stage-2 aromatization reactor 300 which uses “catalyst A” described above.

The second stage reactor 300 operates at about 1 atmosphere pressure and 620° C., while the regenerator 400, which removes the coke formed in the reactor, operates at around 730 C. The heat required for the reaction step is provided by the hot catalyst solid mixture which is preheated during the regeneration step. The second stage reactor 300 converts almost half the ethane fed to it as was the case in performance test 3 in Table 1 above. The effluent from the second stage reactor 300 (stream 3b) is mixed with the effluent from the first stage reactor 100 as described above. Both stage-1 and stage-2 of the aromatization reactor system use a cyclic fixed bed design and their yield structures are based on the performance tests 1 and 3, in Table 1, for stages 1 and 2 respectively.

The liquid products are separated in a sequence of three consecutive columns to obtain the separated liquid products as shown in FIG. 1. The process yields are summarized in Table 2 below. This two-stage mode of operation produces about 7.6 tph of benzene (from column 600 through stream 10), 4.6 tph toluene (from column 700 through stream 11) and 1 tph xylenes (from column 800 through stream 12) resulting in an overall BTX yield of 52.5 wt % and an overall liquid yield of 65 wt % with respect to the propane feed. The undesired fuel gas make (stream 8 from vapor-liquid separator 500) is about 8.7 tph which is about 35 wt % of the mixed feed.

2.2 Two-Stage Process with ethane in the first stage feed

FIG. 1 is a schematic flow diagram for producing aromatics (benzene and higher aromatics) from a feed. This example demonstrates the results of feeding a feedstream containing 19.9 wt % ethane, 80.1 wt % propane using a two stage reactor-regenerator system according to the present invention.

25 tonnes/hr (tph) of mixed feed (stream 1), which constitutes primarily 19.9 wt % ethane and 80.1 wt % propane including minor amounts of methane, butane, etc. (stream 1) are fed to the stage 1 aromatization reactor 100 that uses “Catalyst A” described in example 1. The first stage reactor 100 operates at about 1 atmosphere pressure and at a temperature of about 600° C. while the stage 1 regenerator 200, which removes the coke formed in the reactor 100, operates at around 730° C. The heat required for the reaction step is provided by the hot catalyst solid mixture which is preheated during the regeneration step. The reactor 100 achieves almost complete conversion of propane with a negative value recorded for % ethane conversion as shown in Table 1 for Performance Test 2. This indicates that the amount of ethane made as a byproduct of propane conversion exceeded the amount of ethane converted in this test. The reactor effluent stream 3a is then mixed with the reactor effluent from the second stage reactor 300 (stream 3b), described below. The combined effluent from both the reactor stages (stream 4) is then fed to a separation system where unconverted reactants and light hydrocarbons that consist primarily of ethane and some other hydrocarbons, which may include ethylene, propane, propylene, methane, butane and some hydrogen, are used as the feed (stream 2) for the stage-2 aromatization reactor 300 which uses “catalyst A” described above.

The second stage reactor 300 operates at about 1 atmosphere pressure and 620° C., while the regenerator 400, which removes the coke formed in the reactor, operates at around 730 C. The heat required for the reaction step is provided by the hot catalyst solid mixture which is preheated during the regeneration step. The second stage reactor 300 converts almost half the ethane fed to it as was the case in performance test 3 in Table 1 above. The effluent from the second stage reactor 300 (stream 3b) is mixed with the effluent from the first stage reactor 100 as described above. Both stage-1 and stage-2 of the aromatization reactor system use a cyclic fixed bed design and their yield structures are based on the performance tests 2 and 3, in Table 1, for stages 1 and 2 respectively. The liquid products are separated in a sequence of three consecutive columns to obtain the separated liquid products as shown in FIG. 1. The process yields are summarized in Table 8 below. This two-stage mode of operation produces about 8.3 tph of benzene (from column 600 through stream 10), 4.9 tph toluene (from column 700 through stream 11) and 1 tph xylenes (from column 800 through stream 12) resulting in an overall BTX yield of 56.3 wt % and an overall liquid yield of 70.6 wt % with respect to the mixed feed. The undesired fuel gas make (stream 8 from vapor-liquid separator 500) is about 6.8 tph which is about 29 wt % of the mixed feed.

2.3 Comparison of systems with and without ethane in feed

Table 2 below shows the comparison of the system performance without and with ethane co-feed in the 1^{st} stage of the two stage processes. The processes are compared for similar operating conditions in each of the reactors for the corresponding stages. It is evident from Table 2 that the system with ethane co-feed in the first stage of two-stage operation results in better product yields of benzene, toluene, mixed xylenes and C₉₊ liquids with lower undesired fuel gas make as compared to the one without ethane co-feed. Thus, these results illustrate the benefit of the two-stage lower alkane aromatization process comprised by the present invention, in which ethane is present as a feed component but not converted in the first aromatization stage, and in which ethane byproduct from the first stage is converted in the second aromatization stage.

TABLE 2

| 1^{st} stage Feed | No Ethane | With Ethane |
|---|---|---|
| Feed (wt %) | 0/100/0 | 19.9/80.1/0 |
| (ethane/propane/butane) | | |
| Catalyst | A | A |
| | (stage 1 and 2) | (stage 1 and 2) |
| GHSV (per hr) | 1000 | 1000 |
| Reactor Temp (° C.) | Stage-1: 600 | Stage-1: 600 |
| | Stage-2: 620 | Stage-2: 620 |
| Benzene yield | 30.4% | 33.1% |
| (tonne/tonne_{feed}, %) | | |
| Toluene yield | 18.5% | 19.4% |
| (tonne/tonne_{feed}, %) | | |
| Mixed Xylene yield | 3.6% | 3.7% |
| (tonne/tonne_{feed}, %) | | |
| C9+ liquids yield | 12.6% | 14.3% |
| (tonne/tonne_{feed}, %) | | |
| Total BTX yield | 52.5% | 56.3% |
| (tonne/tonne_{feed}, %) | | |
| Total Liq yield | 65% | 70.6% |
| (tonne/tonne_{feed}, %) | | |
| Total fuel-gas make | 34.6% | 29% |
| (tonne/tonne_{feed}, %) | | |

Note:
All yields are expressed as tonnes of the product per tonne of the mixed feed entering the overall process, expressed as percentage.

### Example 3

In this example the results of laboratory tests are used to represent the improvements in total aromatics yield that may be obtained by using ethane in both stages of the two-stage lower alkane aromatization process of the present invention. The laboratory tests of this example illustrate the aromatics yields that can be obtained with a single-stage aromatization process using a feed consisting of 69.9% wt propane plus 30.1% wt n-butane, a single-stage aromatization process using a feed consisting of 33.2% wt ethane plus 46.8% wt propane plus 20.0% wt n-butane, a two-stage aromatization process in which the first-stage feed consists of 69.9% wt propane plus 30.1% wt n-butane and the second-stage feed consists of the ethane byproduct from the first stage, and the two-stage aromatization process of the present invention in which the first-stage feed consists of 33.2% wt ethane plus 46.8% wt propane plus 20.0% wt n-butane and the second-stage feed consists of the net ethane produced in the first stage.

Fresh 15-cc charges of Catalyst A, prepared as described above in Example 1, were subjected to Performance Tests 4 and 5, which were conducted in the same manner as Performance Tests 1 and 2 described above in Example 1, except that the feed compositions were different as will be described below. Table 3 lists the feed compositions, reactor wall temperatures, and results of online gas chromatographic analyses of the total product streams from Performance Tests 4 and 5, as well as the corresponding data for Performance Test 3 from Example 1 above. Based on composition data obtained from the gas chromatographic analyses, initial ethane, propane, and butane conversions were computed according to the formulas given below:

Ethane conversion, %=100×(% wt ethane in feed−% wt ethane in outlet stream)/(% wt ethane in feed)

Propane conversion, %=100×(% wt propane in feed−% wt propane in outlet stream)/(% wt propane in feed)

Butane conversion, %=100×(% wt butane in feed−% wt C4 in outlet stream)/(% wt butane in feed)

Total propane plus butane conversion, % wt=100×(% wt propane in feed+% wt butane in feed−% wt propane in outlet stream−% wt C4 in outlet stream)/(% wt propane in feed+% wt butane in feed)

For Performance Tests 4 and 5, normalized % wt yields, based on propane plus butane feed converted, for each component, except ethane, in the reactor outlet stream were computed according to the following formula:

Normalized % wt yield of component C=10,000×(% wt component C in reactor outlet stream)/(% wt propane plus butane in feed×% total propane plus butane conversion)

For Performance Tests 4 and 5, the normalized % wt net ethane yield, based on total propane plus butane feed converted, was computed according to the following formula:

Normalized % wt net ethane yield=10,000×(% wt ethane in reactor outlet stream−% wt ethane in feed)/((% wt propane in feed+% wt butane in feed)×% total propane plus butane conversion)

For Performance Test 3, normalized % wt yields, based on ethane feed converted, for each component, except ethane, in the reactor outlet stream were computed according to the following formula:

Normalized % wt yield of component C=100×(% wt component C in reactor outlet stream)/(% wt ethane conversion)

TABLE 3

| | Performance Test No. | | |
|---|---|---|---|
| | 4 | 5 | 3 |
| Reactor Wall Temperature, C. | 600 | 600 | 621 |
| Feed Ethane, % wt | 0 | 33.2 | 100 |
| Feed Propane, % wt | 69.9 | 46.8 | 0 |
| Feed Butane, % wt | 30.1 | 20.0 | 0 |
| Ethane Conversion, % wt | NA | −27.64 | 48.34 |
| Propane Conversion, % wt | 98.8 | 98.57 | NA |
| Butane Conversion, % wt | 99.77 | 99.62 | NA |
| Total Propane + Butane Conversion, % wt | 99.09 | 99.06 | NA |
| Reactor Outlet Composition, % wt | | | |
| Hydrogen | 3.23 | 3.59 | 4.56 |
| Methane | 19.65 | 11.96 | 7.94 |
| Ethylene | 1.84 | 2.47 | 4.37 |
| Ethane | 28.47 | 42.38 | 51.66 |
| Propylene | 0.51 | 0.43 | 0.57 |
| Propane | 0.84 | 0.67 | 0.63 |
| C4 | 0.07 | 0.08 | 0.11 |
| C5 | 0 | 0 | 0 |
| Benzene | 20.48 | 17.29 | 14.61 |
| Toluene | 14.5 | 11.59 | 6.94 |
| C8 Aromatics | 3.1 | 2.49 | 1.19 |
| C9+ Aromatics | 7.31 | 7.05 | 7.41 |
| Total Aromatics | 45.4 | 38.42 | 30.15 |
| Normalized Total Aromatics Yield Based on | 45.82 | 58.06 | |
| Propane + Butane Converted | | | |
| Normalized Ethane Yield Based | 28.73 | 13.87 | |
| on Propane + Butane Converted | | | |
| Normalized Total Aromatics Yield Based | | | 62.37 |
| on Ethane Converted in Stage 2 (Test 3) | | | |
| Added Total Aromatics from Stage 2 | 17.92 | 8.65 | |
| Normalized Total Aromatics Yield, | 63.74 | 66.71 | |
| Stages 1 and 2 | | | |

From the results for Performance Tests 4 and 5 in Table 3, it can be seen that the normalized total aromatics yields obtained, based on total propane plus butane converted, were 45.82% wt for the feed consisting of 69.9% wt propane plus 30.1% wt butane, and 58.06% wt for the feed consisting of 33.2% wt ethane plus 46.8% wt propane plus 20.0% wt n-butane. These results simulate the benefit of including ethane in the feed for a one-stage propane plus butane aromatization process.

From the results for Performance Test 4 in Table 3, it can also be seen that the normalized ethane yield, based on total propane plus butane converted, obtained from the feed consisting of 69.9% wt propane plus 30.1% wt n-butane was 28.73% wt. If all of the ethane byproduct from Performance Test 4 was taken through a second conversion stage, simulated by Performance Test 3 which was conducted with 100% wt ethane feed, then the normalized total aromatics yield obtained from this second stage, based on the original amount of propane plus butane fed to the first stage, would be 17.92% wt. Thus, the total combined normalized aromatics yield, based on original propane plus butane feed, from the first stage fed with 69.9% wt propane plus 30.1% wt n-butane and the second stage fed with byproduct ethane from the first stage would be the sum of 45.82% wt from the first stage plus 17.92% wt from the second stage, for a total normalized aromatics yield of 63.74% wt. These results illustrate the benefit of converting ethane byproduct from the first stage of a C₃-C₄ alkane aromatization process in a second stage, with the second stage providing a substantial increase in the total aromatics yield derived from the original C₃-C₄ alkane feed.

From the results for Performance Test 5 in Table 3, it can be seen that the normalized ethane, based on propane converted, obtained from the feed consisting of 33.2% wt ethane plus 46.8% wt propane plus 20.0% wt n-butane was 13.87% wt. If all of the net ethane byproduct from Performance Test 5 was taken through a second conversion stage, simulated by Performance Test 3 which was conducted with 100% wt ethane feed, then the normalized total aromatics yield obtained from this second stage, based on the original amount of propane plus butane fed to the first stage, would be 8.65% wt. Thus, the total combined normalized aromatics yield, based on original propane plus butane feed, from the first stage fed with 33.2% wt ethane plus 46.8% wt propane plus 20.0% wt n-butane, and the second stage fed with byproduct ethane from the first stage, would be the sum of 58.06% wt from the first stage plus 8.65% wt from the second stage, for a total normalized aromatics yield of 66.71% wt. This 66.71% wt total normalized aromatics yield clearly exceeds the 45.82% wt value obtained from one-stage processing of 69.9% wt propane plus 30.1% wt n-butane feed (simulated by Performance Test 4), the 58.06% wt value obtained from one-stage processing of the feed consisting of 33.2% wt ethane plus 46.8% wt propane plus 20.0% wt n-butane (simulated by Performance Test 5), and the 63.74% wt value obtained from two-stage processing in which the first-stage feed consists of 69.9% wt propane plus 30.1% wt n-butane and the second-stage feed consists of the net ethane byproduct from the first stage (simulated by Performance Tests 5 and 3, respectively). Thus, these results illustrate the benefit of the two-stage lower alkane aromatization process comprised by the present invention, in which ethane is present as a feed component but not converted in the first aromatization stage, and in which ethane byproduct from the first stage is converted in the second aromatization stage.

### Example 4

4.1 Two-Stage Process with no ethane in first stage feed

FIG. 1 is a schematic flow diagram for producing aromatics (benzene and higher aromatics) from a feed. This example demonstrates the results of feeding a feedstream containing 69.9 wt % propane and 30.1 wt % butane using a two stage reactor-regenerator system.

25 tonnes/hr (tph) of feed (stream 1), which constitutes primarily 69.9 wt % propane and 30.1 wt % butane including minor amounts of methane, ethane, butane, etc. (stream 1) is fed to the stage 1 aromatization reactor 100 that uses “Catalyst A” described in example 3. The first stage reactor 100 operates at about 1 atmosphere pressure and at a temperature of about 600° C. while the stage 1 regenerator 200, which removes the coke formed in the reactor 100, operates at around 730° C. The heat required for the reaction step is provided by the hot catalyst solid mixture which is preheated during the regeneration step. The reactor 100 achieves almost complete conversion of propane and butane as shown in Table 3 for Performance Test 3 with a negative conversion for ethane since ethane is made as a byproduct of propane and butane conversion converted in this test. The reactor effluent stream 3a is then mixed with the reactor effluent from the second stage reactor 300 (stream 3b), described below. The combined effluent from both the reactor stages (stream 4) is then fed to a separation system where unconverted reactants and light hydrocarbons that consist primarily of ethane and some other hydrocarbons, which may include ethylene, propane, propylene, methane, butane and some hydrogen, are used as the feed (stream 2) for the stage-2 aromatization reactor 300 which uses “catalyst A” described above.

The second stage reactor 300 operates at about 1 atmosphere pressure and 620° C., while the regenerator 400, which removes the coke formed in the reactor, operates at around 730 C. The heat required for the reaction step is provided by the hot catalyst solid mixture which is preheated during the regeneration step. The second stage reactor 300 converts almost half the ethane fed to it as was the case in performance test 3 in Table 3 above. The effluent from the second stage reactor 300 (stream 3b) is mixed with the effluent from the first stage reactor 100 as described above. Both stage-1 and stage-2 of the aromatization reactor system use a cyclic fixed bed design and their yield structures are based on the performance tests 4 and 3, in Table 3, for stages 1 and 2 respectively.

The liquid products are separated in a sequence of three consecutive columns to obtain the separated liquid products as shown in FIG. 1. The process yields are summarized in Table 4 below. This two-stage mode of operation produces about 7.8 tph of benzene (from column 600 through stream 10), 4.9 tph toluene (from column 700 through stream 11) and 1 tph xylenes (from column 800 through stream 12) resulting in an overall BTX yield of 54.7 wt % and an overall liquid yield of 67.5 wt % with respect to the mixed feed. The undesired fuel gas make (stream 8 from vapor-liquid separator 500) is about 8 tph which is about 32 wt % of the mixed feed.

4.2 Two-Stage Process with ethane in the first stage feed

FIG. 2 is a schematic flow diagram for producing aromatics (benzene and higher aromatics) from a process feed containing 69.9 wt % propane and 30.1 wt % butane using a two stage reactor-regenerator system according to the present invention. However, the feed to the first stage aromatization reactor contains 33.2 wt % ethane, 46.8 wt % propane and 20 wt % butane.

25 tonnes/hr (tph) of mixed feed (stream 1), which constitutes primarily 69.9 wt % propane and 30.1 wt % butane including minor amounts of methane, butane, etc. (stream 1) is blended with a recycle stream (2b) such that the resultant stream (1b) has a composition of 33.2 wt % ethane, 46.8 wt % propane and 20 wt % butane. This resultant mixed stream (1b) is fed to the stage 1 aromatization reactor 100 that uses “Catalyst A” described in example 3. The first stage reactor 100 operates at about 1 atmosphere pressure and at a temperature of about 600° C. while the stage 1 regenerator 200, which removes the coke formed in the reactor 100, operates at around 730° C. The heat required for the reaction step is provided by the hot catalyst solid mixture which is preheated during the regeneration step. The reactor 100 achieves almost complete conversion of propane and butane with a negative value recorded for % ethane conversion as shown in Table 3 for Performance Test 5. This indicates that the amount of ethane made as a byproduct of propane conversion exceeded the amount of ethane converted in this test. The reactor effluent stream 3a is then mixed with the reactor effluent from the second stage reactor 300 (stream 3b), described below. The combined effluent from both the reactor stages (stream 4) is then fed to a separation system where unconverted reactants and light hydrocarbons that consist primarily of ethane and some other hydrocarbons, which may include ethylene, propane, propylene, methane, butane and some hydrogen, are used as the feed (stream 2) for the stage-2 aromatization reactor 300 which uses “catalyst A” described above. The stream 2 is split, as described earlier such that stream 2a is fed to the stage-2 aromatization reactor 300, while the stream 2b is blended with the process feed stream 1 to result in the mixed stream 1b that is fed to the stage-1 reactor.

The second stage reactor 300 operates at about 1 atmosphere pressure and 620° C., while the regenerator 400, which removes the coke formed in the reactor, operates at around 730 C. The heat required for the reaction step is provided by the hot catalyst solid mixture which is preheated during the regeneration step. The second stage reactor 300 converts almost half the ethane fed to it as was the case in performance test 3 in Table 3 above. The effluent from the second stage reactor 300 (stream 3b) is mixed with the effluent from the first stage reactor 100 as described above. Both stage-1 and stage-2 of the aromatization reactor system use a cyclic fixed bed design and their yield structures are based on the performance tests 5 and 3, in Table 3, for stages 1 and 2 respectively.

The liquid products are separated in a sequence of three consecutive columns to obtain the separated liquid products as shown in FIG. 2. The process yields are summarized in Table 4 below. This two-stage mode of operation produces about 8 tph of benzene (from column 600 through stream 10), 5.1 tph toluene (from column 700 through stream 11) and 1.1 tph xylenes (from column 800 through stream 12) resulting in an overall BTX yield of 57 wt % and an overall liquid yield of 70.8 wt % with respect to the mixed feed. The undesired fuel gas make (stream 8 from vapor-liquid separator 500) is about 7.2 tph which is about 28.8 wt % of the mixed feed.

4.3 Comparison of systems with and without ethane

Table 4 below shows the comparison of the system performance without and with ethane co-feed in the 1^{st} stage of the two stage processes. The processes are compared for similar operating conditions in each of the reactors for the corresponding stages. It is evident from Table 4 that the system with ethane co-feed in the first stage of two-stage operation results in better product yields of benzene, toluene, mixed xylenes and C₉₊ liquids with lower undesired fuel gas make as compared to the one without ethane co-feed. Thus, these results illustrate the benefit of the two-stage lower alkane aromatization process comprised by the present invention, in which ethane is present as a feed component but not converted in the first aromatization stage, and in which ethane byproduct from the first stage is converted in the second aromatization stage.

TABLE 4

| 1^{st} stage Feed | No Ethane | With Ethane |
|---|---|---|
| Process Feed (wt %) | 0/69.9/30.1 | 0/69.9/30.1 |
| (ethane/propane/butane) | | |
| Stage-1 reactor Feed (wt %) | 0/69.9/30.1 | 33.2/46.8/20.0 |
| (ethane/propane/butane) | | |
| Catalyst | A | A |
| | (stage 1 and 2) | (stage 1 and 2) |
| GHSV (per hr) | 1000 | 1000 |
| Reactor Temp (° C.) | Stage-1: 600 | Stage-1: 600 |
| | Stage-2: 620 | Stage-2: 620 |
| Benzene yield (tonne/tonne_{feed}, %) | 31.1% | 32.2% |
| Toluene yield (tonne/tonne_{feed}, %) | 19.6% | 20.5% |
| Mixed Xylene yield | 4.0% | 4.3% |
| (tonne/tonne_{feed}, %) | | |
| C9+ liquids yield | 12.8% | 13.8% |
| (tonne/tonne_{feed}, %) | | |
| Total BTX yield (tonne/tonne_{feed}, %) | 54.7% | 57.0% |
| Total Liq yield (tonne/tonne_{feed}, %) | 67.5% | 70.8% |
| Total fuel-gas make | 32.1% | 28.8% |
| (tonne/tonne_{feed}, %) | | |

Note:
All yields are expressed as tonnes of the product per tonne of the mixed feed entering the overall process, expressed as percentage.

1. A process comprising:
a. contacting a lower alkane feed comprising propane and ethane with an aromatization catalyst in a first stage under first stage reaction conditions to produce a first stage product stream comprising ethane and aromatics;
b. separating the aromatics from the first stage product stream to form an aromatics product stream and a second stage feed; and
c. contacting the second stage feed with an aromatization catalyst in a second stage under second stage reaction conditions to produce a second stage product stream comprising ethane and aromatics
d. wherein the amount of ethane in the first stage product stream is equal to from 80 to 300% of the amount of ethane in the lower alkane feed and the amount of ethane in the second stage product stream is equal to at most 80% of the amount of ethane in the second stage feed.

2. The process as claimed in claim 1 wherein the amount of ethane in the first stage product stream is equal to from 150 to 300% of the amount of ethane in the lower alkane feed.

3. The process of claim 1 wherein the amount of ethane in the first stage product stream is equal to from 200 to 300% of the amount of ethane in the lower alkane feed.

4. The process of claim 1 wherein the amount of ethane in the second stage product stream is equal to at most 70% of the amount of ethane in the second stage feed.

5. The process of claim 1 wherein the amount of ethane in the second stage product stream is equal to at most 60% of the amount of ethane in the second stage feed.

6. The process of claim 1 wherein the first stage reaction conditions comprise a temperature of from 400 to 700° C.

7. The process of claim 1 wherein the first stage reaction conditions comprise a temperature of from 480 to 600° C.

8. The process of claim 1 wherein the second stage reaction conditions comprise a temperature of from 400 to 700° C.

9. The process of claim 1 wherein the second stage reaction conditions comprise a temperature of from 575 to 675° C.

10. The process of claim 1 wherein the first stage product stream is produced in at least two reactors aligned in parallel.

11. The process of claim 1 wherein the second stage product stream is produced in at least two reactors aligned in parallel.

12. The process of claim 1 wherein non-aromatic hydrocarbons other than ethane and propane are produced in the first stage.

13. The process of claim 12 wherein the non-aromatic hydrocarbons other than ethane and propane are fed to the second stage as part of the second stage feed.
